(19) United States
(12) Patent Application Publication
(10) Pub. No.: US 2017/0239075 A1
(43) Pub. Date: Aug. 24, 2017

(54) Female Urinal Apparatus

(71) Applicant: HPC Healthline UK Limited, Morden Surrey (GB)

(72) Inventor: Fuad Khan, Morden Surrey (GB)

(21) Appl. No.: 15/515,582

(22) PCT Filed: Sep. 29, 2015
(86) PCT No.: PCT/GB2015/052817

§ 371 Date: Mar. 29, 2017

(30) Foreign Application Priority Data
Sep. 29, 2014 (GB) 1417193.8

(51) Int. Cl. A61F 5/455

(52) U.S. Cl. CPC A61F 5/4556

(57) ABSTRACT

A disposable female urinal comprises a funnel shaped body formed from a moulded fibre material. The body includes a relatively wide receiving mouth at one end narrowing along the length of the body to an outlet opening at the opposing end which forms a spout. The receiving mouth is surrounded by a sealing rim having a shape configured to be pressed against a user's pubic area in use. The rim comprises a curved return edge such that the exposed end surface of the moulded fibre body is directed away from the front end of the device that faces the user in use, ensuring that the edge does not contact the user when the device is pressed against the user in use to improve user comfort.

The present invention relates to female urinal apparatus, and in particular a disposable female urinal device formed of a moulded fibre material.

The direction of female urine flow requires females to be seated during urination. This can create difficulty for women when they are faced with a situations in which they are unable to be seated, for example because a toilet seat is unavailable or sitting on an available toilet seat is undesirable. This situation may occur for example at large events such as music festivals where there are a shortage of toilets and those that are available are typically found in an unsanitary state. In other situations no toilets at all may be available, for example when walking or taking part in other outdoor pursuits. In these situations a woman is required to squat while urinating to ensure that the direction of urine flow does not result in their clothing or shoes becoming wet. The inability to direct urine flow is also problematic for example when attempting to use a bed pan or similar device if bed ridden, as it is often not possible to properly arrange the bed pan at the right location of angle to catch the urine stream, or to predict where the urine stream may initially be directed.

It is therefore know to provide female urination devices for enabling a female to channel and direct their urine flow while standing. A female urination device typically comprises a funnel shaped device formed from a rubber, silicone or plastic material, having a mouth opening that is placed against the user's pubic region and which catches and channels the urine as it is released towards a narrower spouted outlet. By directing the spout the user is able to direct the flow of urine away from their body in a desired directing in a similar manner to male urination. These reusable devices require cleaning and preferably sterilisation after use. However, as the device is typically used in a situation where a toilet is unavailable, it follows that there will rarely be wash facilities immediately at hand to clean the device.

Therefore, disposable female urinal devices have been proposed. Such devices typically are formed from a blank of cardboard that it folded at the point of use for form a cone or funnel. However, whereas a silicon device has a soft feel an dis moulded in an ergonomic form that is comfortable to press against the skin during use, a folded cardboard arrangement presents a sharp straight edges that are not comfortable to press against the body in use. In addition, such disposable devices have been found to be prone to leakage during use.

It is therefore desirable to provide an improved disposable female urinal device which addresses the above described problems and/or which offers improvements generally.

According to the present invention there is provided a disposable female urinal apparatus as described in the accompanying claims.

In an embodiment of the invention there is provided a disposable female urinal apparatus comprising a funnel shaped body formed from a moulded fibre material. The body includes a relatively wide receiving mouth at one end narrowing along the length of the body to an outlet opening at the opposing end which forms a spout. The receiving mouth is surrounded by a sealing rim having a shape configured to be pressed against a user's pubic area in use. Advantageously the rim comprises a curved return edge. The return edge ensures that the exposed end surface of the moulded fibre body is directed away from the front end of eth device that faces the user in use, to ensure that the edge does not contact the user when the device is pressed against the user in use to ensure comfort. In addition, the return edge advantageously presents a curved surface at the contact zone that engages the user for optimum comfort and sealing.

The return edge preferably curves outwardly and returns on itself defining an outer rim wall spaced outwardly from the main body of the funnel.

The rim may include a continuously curved surface or alternatively may include flat sections along all or part of its length.

The mouth section preferably includes an upper end that in use is arranged uppermost relative to the lower end and points up the user's body when engaged. The upper end is narrower than the lower end such that the mouth has a substantially tear drop shape. This shape provides an ergonomic form and a wider base for channelling the urine to the spout end.

The body preferably curves upwardly along its length toward the outlet end. This arrangement has been found to improve the ability to direct the flow of urine from the spout. This arrangement is particularly advantageous for patients lying prone in bed as it enables them to direct urine away from the surface of the bed to a bed pan or urinal.

The return edge is preferably configured such that the exposed end edge moulded fibre body of the body is directed away from the front if the device that contacts the user in use.

The rim preferably has a convex profile along the height of the mouth defined between the upper and lower ends of the mouth top improve the ergonomic fit and sealing of the device.

Preferably the body is formed from a thermoformed or post pressed fibre material which provides the contact surface with a comfortable smooth surface finish to improve comfort, as well as significantly increasing the strength of the apparatus by weight.

The present invention will now be described by way of example only with reference to the following illustrative figures in which:

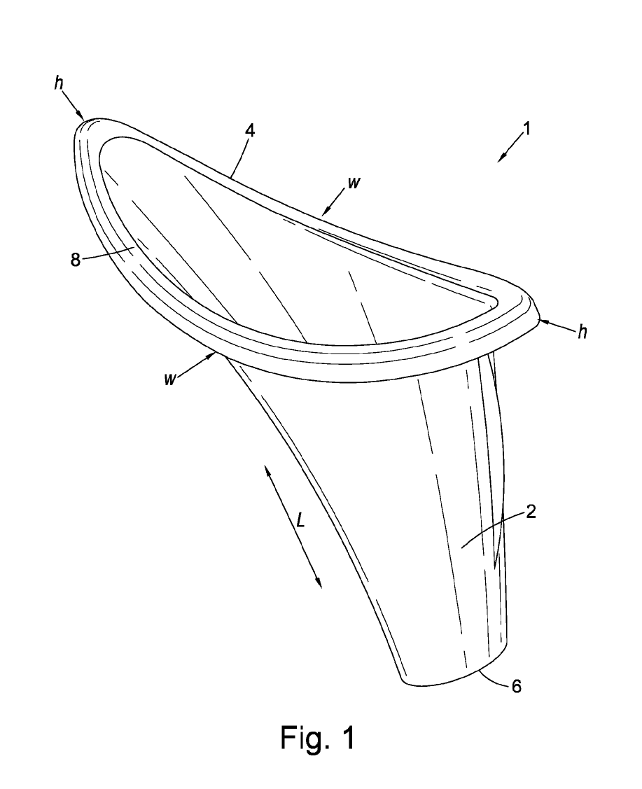

FIG. 1 shows a female urinal device according to an embodiment of the invention.

Referring to FIG. 1 there is provided a female urinal device to enable urination by a female in a standing or prone position. The device includes a body 2 formed from a thermoformed or post pressed fibre material. The body is substantially cylindrical having an inlet opening 4 at a first end and an outlet opening 6 at the opposing end. The inlet opening 4 comprises a reltively wide mouth having a substantially elliptical rim 8 defining a sealing surface for pressing against the pubic area of a user. The mouth 4 is narrower at its upper end 10 than at its base 12 such that the mouth 4 has a substantially tear drop shape. The body 2 tapers radially along its length from the relatively wider mouth 4 to the outlet 6 forming such that the body defines a funnel arrangement. The narrower outlet end 6 defines the spout of the funnel.

The mouth 4 has a width w and a height h. The rim 8 has a convex profile along the height of the mouth 4 to provide it with an ergonomic form for placing against the pubic region of a user. The rim 8 includes a return edge, with the edge being radiussed such that the free exposed edge faces rearwardly in the substantially in the direction of the spout. As such, the rim 8 defines a curved contact surface that is pressed against the user in use to seal the mouth and ensure the urine flow is directed and channelled to the outlet.

Whilst endeavouring in the foregoing specification to draw attention to those features of the invention believed to be of particular importance it should be understood that the Applicant claims protection in respect of any patentable feature or combination of features hereinbefore referred to and/or shown in the drawings whether or not particular emphasis has been placed thereon.

1. A disposable female urinal apparatus comprising a funnel shaped body formed from molded fiber material, the body including a receiving mouth at one end narrowing along the length of the body to an outlet opening at the opposing end, wherein the receiving mouth is surrounded by a sealing rim having a shape configured to be pressed against a user's pubic area in use, and wherein the rim comprises a curved return edge.

2. The disposable female urinal apparatus according to claim 1, wherein the mouth includes an upper end that in use is arranged uppermost relative to the lower end, the upper end being narrower than the lower end such that the mouth has a substantially tear drop shape.

3. The disposable female urinal apparatus according to claim 2, wherein the body curves upwardly along its length toward the outlet end.

4. The disposable female urinal apparatus according to claim 1, wherein the return edge is configured such that the exposed end of the body is directed away from the front if the device that contacts the user in use.

5. The disposable female urinal apparatus according to claim 2, wherein the rim has a convex profile along the height of the mouth defined between the upper and lower ends of the mouth.

6. The disposable female urinal apparatus according to claim 1, wherein the body is formed from a thermoformed or post pressed fiber material.
